(19) United States
(12) Patent Application Publication
(10) Pub. No.: US 2016/0287486 A1
(43) Pub. Date: Oct. 6, 2016

(54) AGENT AND METHOD FOR THE TEMPORARY SHAPING OF KERATIN-CONTAINING FIBRES

(71) Applicant: Henkel AG & Co. KGaA, Duesseldorf (DE)

(72) Inventors: Jisook Baek, Shanghai (CN); Anna Puls, Winsen (DE)

(73) Assignee: Henkel AG & Co. KGaA, Duesseldorf (DE)

(21) Appl. No.: 15/186,270

(22) Filed: Jun. 17, 2016

Related U.S. Application Data
(63) Continuation of application No. PCT/DE2014/200538, filed on Oct. 8, 2014.

(30) Foreign Application Priority Data
Dec. 23, 2013 (DE) 10 2013 227 118.2

(51) Int. Cl. A61K 8/02; A61K 8/81; A61K 8/25; A61Q 5/06; A61K 8/11

(52) U.S. Cl. CPC A61K 8/022; A61Q 5/06; A61K 8/11; A61K 8/25; A61K 8/8158; A61K 2800/56

(57) ABSTRACT

The invention relates to powdered cosmetic compositions containing: a) 10 to 30% by weight of a hydrophobized metal oxide powder, b) 40 to 89% by weight of a polar solvent, and c) 0.01 to 5.0% by weight of an encapsulated fragrance preparation. Said compositions are particularly suitable for reshaping and scenting keratin fibers and provide shine, texture, and silkiness.

### FIELD OF THE INVENTION

The present invention generally relates to the technical field of the temporary reshaping of keratin-containing fibers, in particular human hair.

### BACKGROUND OF THE INVENTION

Styling agents for reshaping keratin-containing fibers have been known for some time and are used in various forms for volumizing, refreshing, and retaining of hairstyles, which for many hair types can only be achieved using setting active substances. Both hair treatment agents used to shape hair permanently and those that shape it temporarily play an important role in this respect. Temporary shaping, which is intended to provide a good hold without impairing the healthy appearance of the hair, such as, for example, the shine thereof, can be achieved, for example, by hairsprays, hair waxes, hair gels, blow-dry waves, etc.

Suitable agents for temporary shaping usually include synthetic polymers as a shaping component. Preparations including a polymer can be applied to the hair by means of propellant gases or by a pump mechanism. Hair gels and hair waxes, on the other hand, are generally applied onto the hair not directly but are distributed in the hair by means of a comb or the hands.

Known forms of temporary styling agents often cannot be dispensed with satisfactory accuracy. Thus, for instance, hair gels, hair creams, and hair waxes are difficult to distribute, once they have been applied to the hair.

Hairsprays can be applied to the hair more uniformly. But because it is not possible for the user to visually determine the total amount of applied styling agent, there is the risk that more styling agent than would actually be necessary is applied to the hair.

Powdered cosmetics are known and have already been used for some time, for instance, in the field of skin treatment. Typical examples, for instance, are makeup powder or eyeshadow. To achieve the powdered consistency, it is necessary to use a powdered carrier material. For instance, a metal oxide such as, e.g., silicon dioxide can be used as a suitable carrier material. Of particular interest is hydrophobized metal oxide or silicon dioxide. This can be obtained, for example, from pyrogenic silicon dioxide, which is commercially obtainable in various specifications. Untreated pyrogenic silicon dioxide carries silanol groups and siloxane groups on the surface. As a result, it has a high affinity for water; i.e., it is hydrophilic. By reaction with suitable organic silicon compounds, alkylsilyl groups can be chemically bound to the surface of the pyrogenic silicon dioxide. Modified silicon dioxide powders are formed that can no longer be wetted by water, i.e., that have hydrophobic properties. This hydrophobized silicon dioxide is suitable for preparing so-called dry water, in which the water droplets are prevented from coalescing again. The resulting powdered solids can have a water content of up to 95%. The enclosed water is released again under mechanical stress, for example, upon being rubbed onto the skin.

Cosmetic or pharmaceutical liquefiable powder compositions are described, for example, in European patent EP 1235554 B1.

The international application WO 03/037287 A1 discloses the use of granules based on pyrogenic silicon dioxide in cosmetic compositions. The special granules can be silanized, i.e., hydrophobized, and are suitable for preparing cosmetic compositions of any consistency, for example, liquids, foams, sprays, or powders.

The international application WO 2007/051511 A1 describes the use of a powdered composition, including 50 to 95% by weight of an aqueous solvent, hydrophobized silicon dioxide powder, and a film-forming and/or setting polymer, present at least in the aqueous solvent, for the temporary reshaping of keratinic fibers.

The subject of the German patent application DE 102008057261 A1 is powdered compositions which are used for the temporary reforming of hair for a very strong hold of the set hairstyle.

The powdered hair cosmetics of the state of the art meanwhile do in fact provide a hold acceptable for hair reforming and are notable for good dispensability. Nevertheless, the result achieved with these agents in regard to the parameters of natural shine and elasticity and hold is also in need of improvement as is their applicability, for example, by application to hands and hair. Lastly, the powdered hair cosmetics known so far are only partially suitable for incorporating oils such as perfume oils, because these reduce the stability of the powdered agents. The fragrance effect of these perfume-containing powders, moreover, lasts only briefly and the fragrance compounds can be applied uniformly to hair only with great difficulty.

The object of the present invention, therefore, was to provide storage-stable powdered hair treatment agents for the temporary reshaping, which are notable for an improved fragrance effect, can be dispensed and applied precisely and simply, do not cause hair to stick together, and give the hair a fuller and natural feel and a natural shine. The durability of the styling result should not be detrimentally affected.

Furthermore, other desirable features and characteristics of the present invention will become apparent from the subsequent detailed description of the invention and the appended claims, taken in conjunction with this background of the invention.

### BRIEF SUMMARY OF THE INVENTION

Powdered cosmetic compositions, including 10 to 30% by weight of a hydrophobized metal oxide powder, 40 to 89% by weight of a polar solvent, and 0.01 to 5.0% by weight of an encapsulated fragrance preparation.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It was determined that powdered cosmetic compositions based on a polyol-emulsifier mixture achieve the aforesaid object. A first subject of the present invention constitutes powdered cosmetic compositions, including
a) 10 to 30% by weight of a hydrophobized metal oxide powder,
b) 40 to 89% by weight of a polar solvent,
c) 0.01 to 5.0% by weight of an encapsulated fragrance preparation.

The powdered compositions of the invention are present preferably in the form of core-shell particles, whose shell includes particles of at least one hydrophobized metal oxide powder and whose liquid core includes an organic polyol and optionally water.

Particles in the context of the invention are particles of solids present as grains (cf. DIN 66160: 1992-09).

Compositions whose particles are freely pourable under their own weight are powdered in the context of the invention (cf. DIN EN ISO 6186: 1998-08).

The powdered compositions of the invention are characterized in that the liquid core is released from the core-shell particles by mechanical stress on the core-shell particles, in particular by friction and/or pressure, and a liquid forms thereby from the powdered composition. Therefore, this is a powdered powder-to-liquid composition. The powdered compositions of the invention can be dispensed very simply. They can be distributed very uniformly in the hair, moreover, because the liquid core is released only under mechanical stress at the site of action and a selective wetting of the hair fibers is enabled. The powder can therefore first be carefully distributed in the hair and only then be stressed more greatly mechanically, for example, by selective massaging of the powder into the hair. As a result, the styling effect is produced only directly on the desired section of hair.

The employed powdered compositions include hydrophobized metal oxide. Preferred compositions are characterized in that they include the hydrophobized metal oxide powder, based on their total weight, in amounts of 10 to 25, preferably of 12 to 22% by weight. The optimum amount in this case depends primarily of the hydrophobicity of the employed silicon dioxide powder. The more hydrophobic the silicon dioxide powder, the less thereof is needed in order to obtain a stable powdered product.

The nature of the hydrophobized metal oxide is in principle not limited, provided it is assured that a powdered product forms upon intensive mixing with the liquid aqueous phase. Metal oxides that have been modified, at least on the surface of the particles, such that the modified particle is wetted less by water than the unmodified particle, are to be understood as hydrophobized in the context of the invention. Silanized, hydrophobized metal oxides are particularly preferred. At least one representative of the group formed by silanes, halosilanes, alkoxysilanes, and silazanes is preferably suitable according to the invention as a reagent for silanizing the metal oxide. Preferably suitable hydrophobized metal oxides of the hydrophobized metal oxide powder are selected according to the invention from at least one representative of the group formed by hydrophobized silicates, hydrophobized aluminum silicates, hydrophobized titanium dioxide, and hydrophobized silicon dioxide. Hydrophobized silicates have proven to be particularly suitable for preparing the cosmetic compositions of the invention, whereby pyrogenic silicic acid aftertreated by silanization or by reaction with polydimethylsiloxane has particular advantages.

The employed powdered compositions preferably include hydrophobized silicon dioxide. The nature of the hydrophobized silicon dioxide is in principle not limited, provided it is assured that a powdered product forms upon intensive mixing with polyol b), copolymer c), and optionally further ingredients.

Particularly preferably, the powdered composition of the invention includes as the hydrophobized metal oxide powder at least silanized, hydrophobized silicon dioxide.

Preferably, at least one representative of the group formed by silanes, halosilanes, alkoxysilanes, and silazanes is suitable according to the invention as a reagent for silanizing the silicon dioxide.

Preferred representatives of the group of silanes are hexa(C₁-C₂₀) alkyl disilanes, particularly hexamethyldisilane.

If a halosilane is used as the silylating agent, selected as the preferred halosilane is at least one compound from the group formed by the compounds
[(C₁-C₂₀)alkyl]_{z′}SiX_{(4-z′})
X₃Si[(CH₂)ₙ—R]
X₂[(C₁-C₂₀)alkyl]Si(CH₂)ₙ—R
[(C₁-C₂₀)alkyl]_{y′+1)}[R+(CH₂)ⁿ]_{(2-y′)}SiX
   where
X denotes a chlorine, bromine, or iodine atom,
z′ is a number 1, 2, or 3,
y′ is a number 0, 1, or 2,
n is an integer from 1 to 20, and
R stands for a group from
(C₁-C₁₀) alkyl, aryl, (C₁-C₆) perfluoroalkyl, —NH₂, —N₃, —SCN, —CH═CH₂,
—O(O)C—C(CH₃)═CH₂, —OCH₂—CH═CH₂,

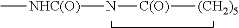

—NH—C(O)O-Me, —NH—C(O)O-Et, —NH—(CH₂)₃—Si(O(C₁-C₆)alkyl)₃.

If an alkoxysilane is used as the silylating agent, selected as the preferred alkoxysilane is at least one compound from the group formed by the compounds
[(C₁-C₂₀)alkylO]_{z}Si(C₁-C₂₀)alkyl_{(4-z)}
[(C₁-C₂₀)alkylO]_{z}Si[(CH₂)ₙ—R]_{(4-z)}
[(C₁-C₂₀)alkylO]₂[(C₁-C₂₀)alkyl]Si(CH₂)ₙ—R
[(C₁-C₂₀)alkylO][(C₁-C₂₀)alkyl]₂Si(CH₂)ₙ—R
[(C₁-C₂₀)alkylO][(C₁-C₂₀)alkyl]Si(CH₂)ₙ—R₂
(C₁-C₂₀alkyl)₃SiO—C(CH₃)═N—Si(C₁-C₂₀)alkyl₃
   where
n is an integer from 1 to 20, and
z denotes a number 1, 2, or 3,
R stands for a group from
(C₁-C₂₀) alkyl, aryl, (C₁-C₆) perfluoroalkyl, —NH₂, —N₃, —SCN, —CH═CH₂,
—O(O)C—C(CH₃)═CH₂, —OCH₂—CH═CH₂,

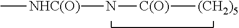

—NH—C(O)O-Me, —NH—C(O)O-Et, —NH—(CH₂)₃-Si(O(C₁—C₆)alkyl)₃.

Selected as the preferred silazane is at least one compound from the class of disilazanes, in particular at least one compound from disilazanes of the formula

R′₂R″Si—NH—SiR′₂R″,
where
R′ denotes a (C₁-C₂₀) alkyl group and
R″ denotes a (C₁-C₂₀) alkyl group or a vinyl group. A particularly preferred silazane is hexamethyldisilazane.

All of the aforesaid alkyl groups, whether (C₁-C₆) alkyl, (C₁-C₁₀) alkyl, or (C₁-C₂₀) alkyl, can be both cyclic and linear or branched. Examples of alkyl groups usable according to the invention are methyl, ethyl, n-propyl, isopropyl, n-butyl, cyclopentyl, cyclohexyl, n-decyl, lauryl, myristyl, cetyl, stearyl, isostearyl, and behenyl.

An example of an aryl group of the invention is the phenyl group.

Examples of a (C₁-C₆) perfluoroalkyl group of the invention are trifluoromethyl, perfluoroethyl, perfluoropropyl, and perfluorohexyl.

Used preferably are hydrophobized silicon dioxides obtained by silanization of pyrogenic silicon dioxide.

Silanized, hydrophobized silicon dioxide are selected particularly preferably from at least one compound of the group formed by trimethyl silylate-coated silicon dioxide, dimethyl silylate-coated silicon dioxide, and octyl silylate-coated silicon dioxide.

Used preferably are hydrophobized silicon dioxides obtained by silanization of pyrogenic silicon dioxide.

A plurality of suitable hydrophobized silicon dioxides are commercially available. Recited as examples are Aerosil® R104 V, Aerosil® R106, Aerosil® R202, Aerosil® R805, Aerosil® R812, Aerosil® R812S, Aerosil® R972, and Aerosil® R8200, all from Degussa, and HDK® H2000, HDK® H2050, and HDK® H3004, all from Wacker.

It is particularly preferred to use the hydrophobized silicon dioxides that are obtainable under the names Aerosil® R202, Aerosil® R812S, or Aerosil® R972. It is very particularly preferred to use the silicon dioxide with the INCI name Silica Silylate, which is marketed by the company Degussa under the name Aerosil® R812S.

Preferred compositions of the invention therefore are characterized in that they include as the hydrophobized metal oxide powder a hydrophobized silicate, preferably a pyrogenic silicic acid aftertreated with polydimethylsiloxane. Suitable metal oxides with the INCI name “Silica Dimethicone Silylate” are marketed, for example, by the company Evonik under the trade name Aerosil® R202.

The particle diameter of the primary particles of preferred hydrophobized metal oxides is preferably less than 5 μm, particularly preferably less than 1 μm, and in particular between 1 and 50 μm.

Preferred furthermore are hydrophobized silicon dioxides that have a specific surface according to BET between 10 and 400 m²/g, preferably between 40 to 300 m²/g, and in particular 80 to 150 m²/g.

The powdered compositions of the invention include a polar solvent as a second essential component. Preferred compositions include the polar solvent, based on their total weight, in amounts of 42 to 88% by weight, preferably of 45 to 87% by weight.

Individual substances or substance mixtures can be used as the polar solvent. Preferred polar solvents, apart from water, are specific polyols. Preferred agents of the invention are notable in that they include less than four, preferably one to three, but in particular only one polyol. Suitable in this case for preparing cosmetic compositions are in particular polyols from the group comprising glycerol, 1,2-ethanediol, polyethylene glycol with MW>400, propanediol, butanediol, particularly 1,3-butanediol, hexanediol, particularly 1,6-hexanediol, sorbitol, threitol, erythritol, arabitol, altritol, ribitol, xylitol, galactitol, mannitol, iditol, and panthenol. Particularly suitable organic polyols are glycerol, sorbitol, and panthenol. These polyols can be converted into a powder form by relatively small amounts of hydrophobized metal oxide powder, usually with less than 10% by weight of metal oxide powder (based on the total weight of the powdered cosmetic composition). Preferred compositions of the invention are therefore characterized in that they include as the organic polyol at least one compound from the group comprising glycerol, sorbitol, and panthenol, preferably glycerol.

In summary, compositions are preferred that include as polar solvent b) at least one solvent from the group comprising water and glycerol, preferably water.

The weight proportion of the polar solvent in general and of the polyol specifically can vary over broad ranges.

Particularly preferred are compositions of the invention that primarily or exclusively include water as the polar solvent. Suitable preferred compositions are characterized in that, based on their total weight, they include
60 to 88% by weight, preferably 65 to 87% by weight of water, and
less than 10% by weight, preferably less than 5% by weight, preferably less than 1% by weight of glycerol.

In the compositions of the invention with a low polyol content, preferably a further polar solvent is added to the compositions by way of compensation, whereby the addition of water is particularly preferred. Preferred compositions are therefore characterized in that, based on their total weight, they include
12 to 30% by weight, preferably 15 to 25% by weight of organic polyol b) and
40 to 75% by weight, preferably 50 to 70% by weight of water.

When a high weight proportion of the polyol is used, the addition of further polar solvents, in particular the addition of water, is omitted as greatly as possible. Suitable compositions preferred according to the invention are characterized in that, based on their total weight, they include
60 to 88% by weight, preferably 65 to 87% by weight of organic polyol b) and
less than 10% by weight, primarily less than 5% by weight, preferably less than 1% by weight of water.

The addition of the polyol improves the mechanical properties of the compositions of the invention during storage and application and has advantageous cosmetic effects.

The compositions of the invention include as a third essential component 0.01 to 5.0% by weight of encapsulated fragrance preparation c). Preferably, the weight proportion of encapsulated fragrance preparation c) in terms of the total weight of the compositions is 0.05 to 3.0% by weight, preferably 0.1 to 2.0% by weight, and in particular of 0.2 to 1.0% by weight.

The addition of encapsulated fragrance preparations, apart from the fragrance effect itself, also improves the applicability and the storage stability of the compositions of the invention.

Individual fragrance compounds, e.g., synthetic products of the ester, ether, aldehyde, ketone, alcohol, and hydrocarbon types, can be used as perfume oils or scents in the context of the present invention. Preferably, however, mixtures of different fragrances are used, which together produce an attractive scent note. Such perfume oils can also include natural fragrance mixtures, as are obtainable from plant sources, e.g., pine, citrus, jasmine, patchouli, rose, or ylang ylang oil.

In order to be perceptible, a fragrance should be volatile, whereby in addition to the nature of the functional groups and the structure of the chemical compound, the molar mass also plays an important role. Therefore, most fragrances possess molar masses of up to approximately 200 daltons, whereas molar masses of 300 daltons and above represent something of an exception. Because of the differing volatility of fragrances, the odor of a perfume or scent made up of multiple fragrances changes during volatilization, whereby the odor impressions are subdivided into a “top note,” “middle note” or “body,” and “end note” (“dry out”). Because the perception of an odor also depends to a large extent on the odor intensity, the top note of a perfume or scent is not made up only of highly volatile compounds, whereas the end note comprises for the most part less volatile, i.e., adherent fragrances. In the composing of perfumes, more volatile fragrances can be bound, for example, to specific fixatives, thereby preventing them from volatilizing too quickly. The subdivision below of fragrances into “more volatile” and “adherent” fragrances therefore makes no statement with regard to the odor impression, and, moreover, as to whether the corresponding fragrance is perceived as a top or middle note.

Compositions of the invention include scents or scent mixtures in encapsulated form. Organic polymers in particular have proven successful as capsule materials for these scents or scent mixtures. Preferred compositions are characterized in that encapsulated fragrance preparation c) comprises a polymer capsule material, preferably a capsule material from the group of anionic or nonionic polysaccharides, in particular a capsule material from the group of sodium starch octenyl succinate and (optionally modified) corn starch.

An optional component of the compositions of the invention are copolymers d), which are obtained by polymerizing the monomers, N-tert-octylacrylamide, acrylic acid, and tert-butylaminoethyl methacrylic acid. Preferred copolymers d) consist of at least 90% by weight, preferably at least 95% by weight, and in particular of at least 97% by weight of the monomers, N-tert-octylacrylamide, acrylic acid, and tert-butylaminoethyl methacrylic acid. Particularly preferred copolymers d) were obtained exclusively from the monomers, N-tert-octylacrylamide, acrylic acid, and tert-butylaminoethyl methacrylic acid.

The copolymers d) described above are marketed, for example, under the name Amphomer® (INCI name: Octylacrylamide/Acrylates/Butylaminoethyl Methacrylate Copolymer) by the company National Starch.

Preferred compositions of the invention are characterized in that the weight proportion of copolymer d) in terms of the total weight of the composition is 0.01 to 15% by weight, preferably 0.02 to 12% by weight, primarily 0.03 to 8.0% by weight, and in particular of 0.05 to 1.0% by weight.

In summary, such compositions are preferred that, based on their total weight, include 0.01 to 15% by weight, preferably 0.02 to 12% by weight, primarily 0.03 to 8.0% by weight, and in particular of 0.05 to 1.0% by weight of at least one copolymer d) from the monomers:
b1) N-tert-octylacrylamide,
b2) acrylic acid,
b3) tert-butylaminoethyl methacrylic acid,
b4) and optionally further monomers.

The composition of some preferred cosmetic agents can be obtained from the following tables (data are given in % by weight, based on the total weight of the cosmetic agent, unless otherwise stated).

| | Formula 1 | Formula 2 | Formula 3 | Formula 4 | Formula 5 |
|---|---|---|---|---|---|
| Hydrophobized metal | 10 to 30 | 10 to 25 | 10 to 25 | 12 to 22 | 12 to 22 |
| oxide powder | | | | | |
| Polar solvent | 40 to 89 | 42 to 88 | 42 to 88 | 45 to 87 | 45 to 87 |
| Encapsulated fragrance | 0.01 to 5.0 | 0.05 to 3.0 | 0.1 to 2.0 | 0.1 to 2.0 | 0.2 to 1.0 |
| Optional additives | To 100 | To 100 | To 100 | To 100 | To 100 |

| | Formula 6 | Formula 7 | Formula 8 | Formula 9 | Formula 10 |
|---|---|---|---|---|---|
| Silica silylates | 10 to 30 | 10 to 25 | 10 to 25 | 12 to 22 | 12 to 22 |
| Polar solvent | 40 to 89 | 42 to 88 | 42 to 88 | 45 to 87 | 45 to 87 |
| Encapsulated fragrance | 0.01 to 5.0 | 0.05 to 3.0 | 0.1 to 2.0 | 0.1 to 2.0 | 0.2 to 1.0 |
| Optional additives | To 100 | To 100 | To 100 | To 100 | To 100 |

| | Formula 11 | Formula 12 | Formula 13 | Formula 14 | Formula 15 |
|---|---|---|---|---|---|
| Silica silylates | 10 to 30 | 10 to 25 | 10 to 25 | 12 to 22 | 12 to 22 |
| Water | 40 to 89 | 42 to 88 | 42 to 88 | 45 to 87 | 45 to 87 |
| Encapsulated fragrance | 0.01 to 5.0 | 0.05 to 3.0 | 0.1 to 2.0 | 0.1 to 2.0 | 0.2 to 1.0 |
| Optional additives | To 100 | To 100 | To 100 | To 100 | To 100 |

| | Formula 16 | Formula 17 | Formula 18 | Formula 19 | Formula 20 |
|---|---|---|---|---|---|
| Silica dimethicone | 10 to 30 | 10 to 25 | 10 to 25 | 12 to 22 | 12 to 22 |
| silylates | | | | | |
| Polar solvent | 40 to 89 | 42 to 88 | 42 to 88 | 45 to 87 | 45 to 87 |
| Encapsulated fragrance | 0.01 to 5.0 | 0.05 to 3.0 | 0.1 to 2.0 | 0.1 to 2.0 | 0.2 to 1.0 |
| Optional additives | To 100 | To 100 | To 100 | To 100 | To 100 |

| | Formula 21 | Formula 22 | Formula 23 | Formula 24 | Formula 25 |
|---|---|---|---|---|---|
| Silica dimethicone | 10 to 30 | 10 to 25 | 10 to 25 | 12 to 22 | 12 to 22 |
| silylates | | | | | |
| Water | 40 to 89 | 42 to 88 | 42 to 88 | 45 to 87 | 45 to 87 |
| Encapsulated fragrance | 0.01 to 5.0 | 0.05 to 3.0 | 0.1 to 2.0 | 0.1 to 2.0 | 0.2 to 1.0 |
| Optional additives | To 100 | To 100 | To 100 | To 100 | To 100 |

| | Formula 26 | Formula 27 | Formula 28 | Formula 29 | Formula 30 |
|---|---|---|---|---|---|
| Hydrophobized metal | 10 to 30 | 10 to 25 | 10 to 25 | 12 to 22 | 12 to 22 |
| oxide powder | | | | | |
| Polar solvent | 40 to 89 | 42 to 88 | 42 to 88 | 45 to 87 | 45 to 87 |
| Encapsulated fragrance | 0.01 to 5.0 | 0.05 to 3.0 | 0.1 to 2.0 | 0.1 to 2.0 | 0.2 to 1.0 |
| Copolymer c) | 0.01 to 15 | 0.02 to 12 | 0.03 to 8.0 | 0.05 to 1.0 | 0.05 to 1.0 |
| Optional additives | To 100 | To 100 | To 100 | To 100 | To 100 |

| | Formula 31 | Formula 32 | Formula 33 | Formula 34 | Formula 35 |
|---|---|---|---|---|---|
| Silica silylates | 10 to 30 | 10 to 25 | 10 to 25 | 12 to 22 | 12 to 22 |
| Polar solvent | 40 to 89 | 42 to 88 | 42 to 88 | 45 to 87 | 45 to 87 |
| Encapsulated fragrance | 0.01 to 5.0 | 0.05 to 3.0 | 0.1 to 2.0 | 0.1 to 2.0 | 0.2 to 1.0 |
| Copolymer c) | 0.01 to 15 | 0.02 to 12 | 0.03 to 8.0 | 0.05 to 1.0 | 0.05 to 1.0 |
| Optional additives | To 100 | To 100 | To 100 | To 100 | To 100 |

| | Formula 36 | Formula 37 | Formula 38 | Formula 39 | Formula 40 |
|---|---|---|---|---|---|
| Silica silylates | 10 to 30 | 10 to 25 | 10 to 25 | 12 to 22 | 12 to 22 |
| Water | 40 to 89 | 42 to 88 | 42 to 88 | 45 to 87 | 45 to 87 |
| Encapsulated fragrance | 0.01 to 5.0 | 0.05 to 3.0 | 0.1 to 2.0 | 0.1 to 2.0 | 0.2 to 1.0 |
| Copolymer c) | 0.01 to 15 | 0.02 to 12 | 0.03 to 8.0 | 0.05 to 1.0 | 0.05 to 1.0 |
| Optional additives | To 100 | To 100 | To 100 | To 100 | To 100 |

| | Formula 41 | Formula 42 | Formula 43 | Formula 44 | Formula 45 |
|---|---|---|---|---|---|
| Silica dimethicone | 10 to 30 | 10 to 25 | 10 to 25 | 12 to 22 | 12 to 22 |
| silylates | | | | | |
| Polar solvent | 40 to 89 | 42 to 88 | 42 to 88 | 45 to 87 | 45 to 87 |
| Encapsulated fragrance | 0.01 to 5.0 | 0.05 to 3.0 | 0.1 to 2.0 | 0.1 to 2.0 | 0.2 to 1.0 |
| Copolymer c) | 0.01 to 15 | 0.02 to 12 | 0.03 to 8.0 | 0.05 to 1.0 | 0.05 to 1.0 |
| Optional additives | To 100 | To 100 | To 100 | To 100 | To 100 |

| | Formula 46 | Formula 47 | Formula 48 | Formula 49 | Formula 50 |
|---|---|---|---|---|---|
| Silica dimethicone | 10 to 30 | 10 to 25 | 10 to 25 | 12 to 22 | 12 to 22 |
| silylates | | | | | |
| Water | 40 to 89 | 42 to 88 | 42 to 88 | 45 to 87 | 45 to 87 |
| Encapsulated fragrance | 0.01 to 5.0 | 0.05 to 3.0 | 0.1 to 2.0 | 0.1 to 2.0 | 0.2 to 1.0 |
| Copolymer c) | 0.01 to 15 | 0.02 to 12 | 0.03 to 8.0 | 0.05 to 1.0 | 0.05 to 1.0 |
| Optional additives | To 100 | To 100 | To 100 | To 100 | To 100 |

The cosmetic compositions of the invention can include further auxiliary and care substances and additives. The weight proportion of the further ingredients, included in the powdered compositions of the invention, apart from components a) to c), in particular the further auxiliary and care substances and additives included in these compositions, in terms of the total weight of the powdered compositions of the invention is preferably less than 10% by weight, preferably less than 5.0% by weight, particularly preferably less than 2.0% by weight, and in particular less than 1.0% by weight. The weight proportion of these auxiliary and care substances and additives in the total weight of the cosmetic agents of the invention can be, for example, 0.001 to 2% by weight, in particular 0.01 to 0.5% by weight.

The powdered compositions of the invention can be packaged in any containers, provided it is assured that the mechanical stress on the powder during the removal of the composition does not lead to liquefaction. Suitable are, for example, jars, bottles, or Tetra Paks as well, whereby the container can be designed, for example, with a pouring and dispensing means.

A further subject of the present invention is the use of a cosmetic composition of the invention for the temporary reshaping of keratin-containing fibers, in particular human hair. When the powdered composition is used for the temporary reshaping of keratinic fibers, preferably first the desired quantity of powdered composition is removed from the container. The composition can be applied directly onto the keratinic fibers to be treated or else, for example, onto one's hand. In the first case, the applied powder can be exposed to a mechanical stress directly on the keratinic fibers, for example, by means of one's hands, with the result that the liquid aqueous phase is released directly onto the fibers. If the powdered composition is first placed onto one's hand, it can then first be carefully distributed in the hair and only then in turn be subjected to greater mechanical stress, for example, by controlled massaging of the powder into the hair. The liquid aqueous phase is thereby released on the hair.

It is, of course, also possible to rub the powdered composition on one's hand and only then to apply the resulting liquid or pasty agent to the keratinic fibers. This procedure is not preferred, however, because an essential advantage of the powdered consistency of the styling agent, namely, good distribution capability, is thereby sacrificed. The powdered composition can, of course, also be applied using an aid, for example, a brush, a sponge, cloth, a hairbrush, or a comb.

A further subject of the present invention is a method for the temporary reshaping of keratin-containing fibers, in particular human hair, in which method the keratinic fibers are acted upon by a cosmetic composition of the invention and their shape is temporarily retained, characterized in that before, during, or after application to the keratinic fibers, a plastically deformable mass is formed from the cosmetic composition by the action of a force.

### EXAMPLES

The powdered styling agents V1 to V6 were prepared as described below (quantities are given in % by weight):

| | V1 | V2 | V3 | V4 | V5 | V6 |
|---|---|---|---|---|---|---|
| Silica silylates | 15 | 19 | 19 | 20 | 20 | 20 |
| Encapsulated | 0.2 | 0.3 | 0.2 | 0.2 | 0.3 | 0.2 |
| fragrance | | | | | | |
| Amphomer * | 0.05 | 0.05 | — | 0.05 | 0.05 | — |
| Water | To 100 | To 100 | To 100 | To 100 | To 100 | To 100 |

* Amphomer ® (INCI name: Octylacrylamide/Acrylates/Butylaminoethyl Methacrylate Copolymer) from the company National Starch.

A stable powder was formed by mixing of the components. The thus obtained finished styling powder was filled into polyethylene bottles.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. Powdered cosmetic composition, comprising:
a) 10 to 30% by weight of a hydrophobized metal oxide powder,
b) 40 to 89% by weight of a polar solvent,
c) 0,01 to 5.0% by weight of an encapsulated fragrance preparation.

2. The composition according to claim 1, wherein the hydrophobized metal oxide powder, comprises 10 to 25 wt % of the total cosmetic composition.

3. The composition according to claim 1, wherein the hydrophobized metal oxide power comprises 12 to 22 wt % of the total cosmetic composition.

4. The composition according to claim 1, wherein the hydrophobized metal oxide powder is a hydrophobized silicate obtained by silanization of pyrogenic silicon dioxide.

5. The composition according to claim 1, characterized in that, based on its total weight, it includes 42 to 88% wt % of polar solvent b).

6. The composition according to claim 1, wherein the polar solvent b) is selected from the group consisting of water and glycerol.

7. The composition according to claim 1, wherein the polar solvent b) includes water.

8. The composition according to claim 1, wherein the composition comprises, based on its total weight, 60 to 88% by weight water and less than 10 wt % of glycerol.

9. The composition according to claim 1, wherein the composition comprises, based on its total weight, 65 to 87% by weight of water and less than 5 wt % glycerol.

10. The composition according to claim 1, wherein the composition comprises, based on its total weight, 12 to 30 wt % organic polyol and 40-75 wt % water.

11. The composition according to claim 1, wherein the composition comprises, based on its total weight, 15 to 25% organic polyol and 50 to 70% water.

12. The composition according to claim 10, wherein the organic polyol is glycerol.

13. The composition according to claim 1, wherein the composition comprises, based on its total weight, 60-88 wt % organic glycerol and less than 10 wt % water.

14. The composition according to claim 1, wherein the composition comprises, based on its total weight, 65-87 wt % organic polyol and less than 5 wt % water.

15. The composition according to claim 1, wherein the encapsulated fragrance preparation c) comprises 0.05 to 3.0 wt % of the total composition.

16. The composition according to claim 1, wherein the encapsulated fragrance preparation c) comprises 0.1 to 2.0% of the total composition.

17. The composition according to claim 1, wherein the encapsulated fragrance preparation c) comprises a polymer capsule material.

18. The composition according to claim 1, wherein the encapsulated fragrance preparation c) comprises a capsule material includes anionic or nonionic polysaccharides.

19. The composition according to claim 1, wherein the composition comprises, based on its total weight, 0.01 to 15% by weight of at least one copolymer from the monomers
b1) N-tert-octylacrylamide,
b2) acrylic acid,
b3) tert-butylaminoethyl methacrylic acid,
b4) and optionally further monomers.

20. A method for the temporary shaping of keratin-containing fibers, in particular human hair, in which the keratinic fibers are acted upon by a cosmetic composition according to one of claim 1 and their shape is temporarily retained, characterized in that before, during, or after application to the keratinic fibers, a plastically deformable mass is formed from the cosmetic composition by the action of a force.
